(19) United States
(12) Patent Application Publication
(10) Pub. No.: US 2006/0008799 A1
(43) Pub. Date: Jan. 12, 2006

(54) Rapid haplotyping by single molecule detection

(76) Inventors: Hong Cai, Los Alamos, NM (US); Peter M. Goodwin, Los Alamos, NM (US); Richard A. Keller, Los Alamos, NM (US); James H. Werner, Los Alamos, NM (US)

Correspondence Address: UNIVERSITY OF CALIFORNIA;LOS ALAMOS NATIONAL LABORATORY, P.O. BOX 1663, MS A187, LOS ALAMOS, NM, 87545, US

(21) Appl. No.: 09/862,855

(22) Filed: May 21, 2001

Related U.S. Application Data
(60) Provisional application No. 60/206,512, filed on May 22, 2000.

(51) Int. Cl. C12Q 1/68

(52) U.S. Cl. 435/6

(57) ABSTRACT

The present invention includes a method for rapid haplotyping a DNA or RNA segment. Two or more target sites on a segment of DNA or RNA are labeled with separate distinguishable luminescent hybridization probes, where the targets are selected genetic markers. A dilute solution is formed containing the labeled DNA or RNA segments. Each DNA or RNA segment is illuminated with light beams effective to excite each luminescent hybridization probe, when present. The presence or absence of each luminescent hybridization probe on each DNA or RNA segment is detected to determine the haplotype of each DNA or RNA segment.

### RELATED CASES

This case claims the benefit of U.S. Provisional Application 60/206,512, filed May 22, 2000.

### STATEMENT REGARDING FEDERAL RIGHTS

This invention was made with government support under Contract No. W-7405-ENG-36 awarded by the U.S. Department of Energy. The government has certain rights in the invention.

### SEQUENCE LISTING

The DNA sequences listed herein are submitted on the single compact disk submitted herewith.

### FIELD OF THE INVENTION

The present invention relates generally to haplotyping, and, more particularly, to rapid haplotyping using single molecule fluorescence detection.

### BACKGROUND OF THE INVENTION

As the Human Genome Project progresses rapidly toward completion of the human DNA sequence, it is recognized that natural sequence variation (i.e., polymorphisms) is a fundamental property of all genomes. Any two human chromosomes (haploids) show multiple sites and types of polymorphisms. Some polymorphisms have functional implications and cause or implicate disease, whereas many do not.

Polymorphisms exist in different forms such as single nucleotide variations [nucleotide repeats, multibase deletion (more than one nucleotide deleted from the consensus sequence), multibase insertion (more than one nucleotide inserted from the consensus sequence)], microsatellite repeats (small numbers of nucleotide repeats with a typical 5-1000 repeat units), di-nucleotide repeats, tri-nucleotide repeats, sequence rearrangements (including translocation and duplication), chimeric sequence (two sequences from different gene origins are fused together), and the like. Among all these sequence polymorphisms, the most frequent polymorphisms in the human genome are single-base variations, also called single-nucleotide polymorphisms (SNPs). SNPs are abundant, stable and widely distributed across the genome. SNPs are important for the following reasons:

(1) SNPs serve as ideal genetic markers for complex disease association studies (linkage analysis), in which particular alleles (one of two or more genes that occur at the same locus of homologous chromosomes) of genetic markers (haplotype) in close proximity to a disease mutation are consistently associated with the disease. Recently, a collaborative effort between public and private sectors has been undertaken to produce a large library of ˜300,000 SNPs in the human genome. It is expected that the Human Genome Project, coupled with high throughput SNP screening and genotyping systems, will permit the rapid establishment of this database. The availability of the SNP library as well as other genetic polymorphisms will allow the elucidation of complex genetic components of human disease and thereby accelerate the drug discovery process (Brookes 1999; Kowk 1999).

(2) SNPs are good markers for population, evolution and forensic studies.

(3) Polymorphism profiles offer the potential to assess a disease risk or predict a drug response based on an individual's genetic profile.

(4) SNP profiles may be used to tailor drug treatments to individual patients, to improve the efficacy and safety of the treatment.

As the first step toward linking SNP profiles for diagnostic and medical treatment, the linkage between a disease and a response to a particular set of polymorphism (or SNPs) has to be established. In such linkage or association studies, a group of patients with a particular disease is compared to a control group to find genetic factors that occur significantly more (or less) frequently in the affected individuals than in the control. The study is typically done with the help of well-mapped polymorphism markers. It is possible to map disease genes to particular regions on chromosomes by studying the transmission of genetic markers along with a disease since the markers in a close physical proximity to a disease mutation co-segregate with the disease at a higher frequency than the more distant markers. Due to the abundance (estimated to be one of every one thousand bases) and stability of SNPs, they serve as good genetic markers for association studies. The effort to build a large SNP library is mainly motivated by the prospect of using SNPs as genetic markers for disease association studies. It has been proposed that an average of 5 SNPs be used for each candidate disease gene in association studies. About 40,000 genes in a human cell, or about 200,000 SNPs, will be needed for such association studies.

The haplotype is a set of genetic determinants located on a single chromosome and it typically contains a particular combination of alleles (all the alternative sequences of a gene) in a region of a chromosome. In other words, the haplotype is a phased sequence information on individual chromosomes. Very often, phased SNPs on a chromosome define a haplotype. The combination of two haplotypes on two human chromosomes ultimately determines the genetic profiles of a human cell. It is the haplotype that determines a linkage between a specific genetic marker and a disease mutation (Kowk 1999; Davidson 2000). Current methods of scoring SNPs (such as hybridization microarray or direct gel sequencing reviewed by Landgren 1998) can accurately type individual SNPs, but can not determine which chromosome of a diploid pair is associated with each polymorphism. Without the phased information of haplotypes, it might be impossible to detect the association due to numerous possibilities of different haplotypes (Hodge 1999; Kowk 1999; Drysdale 2000).

The haplotype deduced from a genotype is typically done in bulk as follows (inferred haplotypes). The region of interest is PCR amplified, the genotype is determined, and the haplotype is deduced from homozygous individuals. Since the genotype is based on a bulk measurement on a mixture of both chromosomes, this genotyping approach has serious limitations for large numbers of SNP markers. Consider the following example:

-----------A1---------B2---------C1---------M---------D1-----------E2
-----------A2---------B1---------C2-------------------D2-----------E1

For a given five SNP genotype results, A1/A2-B1/B2-C1/C2-D1/D2-E1/E2, there are 16 possible haplotypes (Kowk 1999), as shown in Table 1.

TABLE 1

| | A1/B1/C1/D1/E1 and A2/B2/C2/D2/E2 |
|---|---|
| 2 | A1/B1/C1/D1/E2 and A2/B2/C2/D2/E1 |
| 3 | A1/B1/C1/D2/E1 and A2/B2/C2/D1/E2 |
| 4 | A1/B1/C2/D1/E1 and A2/B2/C1/D2/E2 |
| 5 | A1/B2/C1/D1/E1 and A2/B1/C2/D2/E2 |
| 6 | A2/B1/C1/D1/E1 and A1/B2/C2/D2/E2 |
| 7 | A1/B2/C2/D1/E1 and A2/B1/C1/D2/E2 |
| 8 | A1/B2/C1/D2/E1 and A2/B1/C2/D1/E2 |
| 9 | A1/B2/C1/D1/E2 and A2/B1/C2/D2/E1 |
| 10 | A1/B1/C2/D1/E2 and A2/B2/C1/D2/E1 |
| 11 | A1/B1/C2/D2/E1 and A2/B2/C1/D1/E2 |
| 12 | A1/B1/C1/D2/E2 and A2/B2/C2/D1/E1 |
| 13 | A1/B1/C2/D2/E2 and A2/B2/C1/D1/E1 |
| 14 | A1/B2/C2/D2/E1 and A2/B1/C1/D1/E2 |
| 15 | A1/B2/C2/D1/E2 and A2/B1/C1/D2/E1 |
| 16 | A1/B2/C1/D2/E2 and A2/B1/C2/D1/E1 |

For a 5-SNP mapping on a gene, there are 528 possible haploidtypes, and for a given genotype, there may be as many as 16 different haploidtypes. As the number of SNP markers grows larger, the identification of the haploidtype from the genotype is increasingly difficult since the number of possible haploidtypes increases rapidly, and the probability of finding a homozygous individual decreases. Without knowing the particular haplotypes, the mutation (M) association to the nearby C1 or D1 might be missed.

Traditionally, association studies have been successful only for simple, monogenic diseases involving a small number of markers, where the possible combinations of different haplotypes are limited. Therefore, the haplotypes can be typically deduced from genotypes by typing many individuals and by the availability of homozygotes and parental information. However, most diseases are complex and involve multiple genes. For polygenic association studies, many more markers are needed and, therefore, the number of possible haplotypes is large. In these cases, it is extremely difficult to infer the haplotype from the genotype. Many sophisticated algorithms have been developed for haplotype prediction and they are typically 70-90% accurate. Such accuracy is not useful when typing a large numbers of SNPs and also is not acceptable for clinical diagnostic purposes. In addition, it is often impossible or impractical to obtain parental genomic DNA. This raises a serious challenge: there is no easy way to directly determine a haplotype except when it is on the sex chromosomes where X and Y chromosome are sufficiently different to be distinguished in bulk methods.

As shown in FIG. 1, a genetic profile based on a genotype can be incomplete, because it fails to provide the locations of SNPs on two chromosomes. For example, consider two genetic markers (or SNPs), A and B, on the same gene. For a genotype of aA/bB (A and B presents the wild type or dominant genotype that naturally occurs; a and b represent two mutations.), there are two possible combinations of haplotypes, ab/AB and Ab/aB. The disease phenotype for the individual with ab/AB may be less severe compared to the individual with Ab/aB. This is because the individual with ab/AB has one intact copy of the gene, whereas the individual with Ab/aB has no intact copy on either chromosome. For cases like this, the ability to find out whether two mutations are on the same chromosome or on different chromosomes (haplotypes) in a routine clinical setting is particularly useful for future risk assessment and disease diagnostics.

Another conventional alternative for haplotyping is allele-specific polymerase chain reaction (allele-specific PCR (Ruano 1990)), which is the most commonly used method for direct haplotyping. In these reactions, SNP-specific PCR primers are designed to distinguish and amplify a specific haplotype from two chromosomes. Such reactions require stringent reaction conditions and individual optimization for each target. Therefore, this approach is not suitable for a large scale and high throughput haplotyping. More importantly, such assays are subject to the length limitations of PCR amplification and are not capable of typing SNPs that are more than several kilobases (kb) apart. In addition, such an amplification-based typing is often complicated by the contamination of a small amount of genomic DNA other than the sample DNA during sample handling process

Other haplotyping methods include single sperm or single chromosome measurements (Ruano 1990; Zhang 1992; Vogelstein 1999). In a single sperm sorting assay, PCR amplified DNA from individual sorted sperm cells is genotyped. Multiple sperm cells (at least 3-5) from an individual are typed in order to have enough statistical confidence to reveal the two haplotypes. In principle, this sorting approach could be applied to chromosomes. However, this technique is complicated, and, so far, has been successful in only a few research labs.

The molecular cloning method clones a target region of an individual's DNA (or cDNA) into a vector, and genotypes the DNA obtained from single colonies. For each individual, multiple colonies are needed to obtain two haplotypes. This method has been used by many laboratories, but is very labor-intensive, time-consuming and can be difficult to perform in some cases. Researchers are forced to use it because there is no easy alternatives.

Finally, haplotyping by AFM (Atomic Force Microscopy) imaging (Woolley et al. 2000, Taton 2000) is a new approach to directly visualize the polymorphic sites on individual DNA molecules. This method utilizes AFM with high resolution single walled carbon nanotube probes to read directly multiple polymorphic sites in DNA fragments containing from 100-10,000 bases. This approach involves specific hybridization of labeled oligonucleotide probes to target sequences in DNA fragments followed by direct reading of the presence and spatial localization of the labels by AFM. However, the throughput and sensitivity of such systems remain to be demonstrated; currently 200 samples per day, each with 10 images, can be processed.

In summary, there is no easy way to determine a haplotype currently except by using the sex chromosomes. All of the conventional methods require DNA amplification by PCR or cloning and extensive optimization. They are slow, labor intensive and expensive to perform, and not suited for a large-scale association studies, or routine clinical diagnostics (Kowk 1999).

Various objects, advantages and novel features of the invention will be set forth in part in the description which follows, and in part will become apparent to those skilled in the art upon examination of the following or may be learned by practice of the invention. The objects and advantages of the invention may be realized and attained by means of the instrumentalities and combinations particularly pointed out in the appended claims.

### SUMMARY OF THE INVENTION

The present invention includes a method for rapid haplotyping a DNA or RNA segment. Two target sites on a segment of DNA or RNA are labeled with separate distinguishable luminescent hybridization probes, where the target sites are selected genetic markers. The presence or absence of each luminescent hybridization probe on each DNA or RNA segment is detected to determine the haplotype of each DNA or RNA segment.

In one embodiment, a dilute solution is formed containing the labeled DNA or RNA segments. Each labeled DNA or RNA segment is illuminated with light beams effective to excite each luminescent hybridization probe, when present.

### BRIEF DESCRIPTION OF THE DRAWINGS

The accompanying drawings, which are incorporated in and form a part of the specification, illustrate embodiments of the present invention and, together with the description, serve to explain the principles of the invention. In the drawings:

FIG. 1 illustrates haplotyping in accordance with one aspect of the present invention.

FIG. 2 schematically depicts an exemplary apparatus for rapid haplotyping by two color single molecule fluorescence detection.

FIGS. 3A, B, and C depict the detection by fluorescence correlation spectroscopy of two color labels on a single DNA target.

### DETAILED DESCRIPTION

The present invention for rapid haplotyping uses a single molecule approach based on the simultaneous detection of two luminescent labels that are specific to neighboring genetic markers, such as SNPs, from single chromosomes. Suitable techniques for distinguishing luminescence include color differentiation, luminescence lifetime, and luminescence intensity. By using single molecule detection and identification, the co-location of two markers on a given haploid can be rapidly determined.

The present invention encompasses the following types of targets, distinguishable luminescent hybridization probes, and labeling strategies, each of which is individually well known, but not previously applied to haplotyping:
(a) Targets of haplotyping are all sorts of DNA and RNA variations that are necessary to define a haplotype. They include any DNA or mRNA (cDNA) targets such as single nucleotide polymorphisms, single nucleotide variations [nucleotide repeats, multibase deletion (more than one nucleotide deleted from the consensus sequence), multibase insertion (more than one nucleotide inserted from the consensus sequence)], microsatellite repeats (small numbers' of nucleotide repeats with a typical 5-1000 repeat units), di-nucleotide repeats, tri-nucleotide repeats, sequence rearrangements (including translocation and duplication), chimeric sequence (two sequences from different gene origins are fused together), and the like. Samples that contain the targets can be unamplified genomic DNA or RNA samples or amplified DNA (or cDNA).
(b) Target labels are two sets of distinguishable luminescent hybridization probes that may be identified by difference in color, luminescence lifetime, luminescence intensity, luminescence burst duration, and luminescence polarization anisotropy. Such luminescent labels can be single dye molecules, energy transfer dye pairs, nano-particles, luminescent nano-crystals (e.g., quantum dots), intercalating dyes or molecular beacon that only fluoresce upon binding to a target. The forms of the hybridization probes can be any of DNA, cDNA, RNA, PNA (peptide nucleic acid) or LNA (locked nucleic acid) in either natural or synthetic or mixed use of any above forms.
(c) The design strategies for target-specific hybridization probes can be generalized into a one probe approach and multiple probes such as a two-probe approach (Landegren 1998). (1) One probe labeling strategy with probes that are specific for the targets, especially for SNP targets: one hybridization oligo DNA, cDNA, RNA, beacon, PNA, LNA or artificial mismatched oligo or other chemically modified and chimeric oligos (nondenaturation approaches, too). (2) Two or more oligo probes that act together to identify a target: hybridization pair, invader oligo pair, ligation oligo pair, mismatch extension, 5′-exonuclease oligo pair, 3′-exonuclease pair. Oligos can be DNA, cDNA, RNA, PNA, LNA, beacon or other modified and chimeric oligos.
(d) The specificity of probes will be evaluated on a commercial flow cytometer using the known protocols (Cai 1998; Cai 2000).
(e) The concentration range of samples for measurement can range from 100 nanomolar (nM) to the sub femtomolar (fM) range. Therefore, samples can be measured over a wide concentration range. This is especially useful for a very dilute sample in a routine clinical practice. A concentrated sample can be diluted to a proper range for detection.

Conventional fluorescence/luminescence characteristics and single molecule detection apparatus are used to provide the ultrasensitive fluorescence detection methods used in the present invention. The ability to detect and identify single luminescent molecules as they cross a focused excitation laser beam is now an available research tool. See, e.g., U.S. Pat. No. 6,049,380, issued Apr. 11, 2000; U.S. Pat. No. 5,834,204, issued Nov. 10, 1998; U.S. Pat. No. 5,827,663, issued Oct. 27, 1998; U.S. Pat. No. 5,799,682, issued Sep. 1, 1998, all incorporated herein by reference. Work at Los Alamos National Laboratory has led to many of the advances in this field, which are reviewed in a number of recent papers (Goodwin, Ambrose et al. 1996; Keller, Ambrose et al. 1996; Ambrose, Goodwin et al. 1999), incorporated herein by reference.

The following fluorescence/luminescence parameters can be measured at the single molecule level (Ambrose et al. 1999):

Luminescence emission spectral distribution (color).

Luminescence emission decay rate (lifetime).

Luminescence burst intensity (burst size).

Luminescence burst duration.

Luminescence polarization anisotropy.

All of these can be used individually or in combination to distinguish between labeled hybridization probes and to determine the presence of one or more labeled hybridization probes on a target fragment.

Measurements may be made on individual single molecule luminescence bursts to determine the presence of one or more labeled hybridization probes on a target fragment. Such measurements require that the luminescent molecules be interrogated one-at-a-time, that is, the average occupancy of the probe volume is much less than one luminescent molecule. Alternatively, fluorescence correlation spectroscopy (FCS) can be used to characterize fluctuations in the time history of the fluorescence (Rigler et al. 1993). For example, fluorescence from a single detection channel can be auto-correlated to measure the average fluorescence burst shape and duration for fluorescent molecules diffusing across the interrogation volume. The cross-correlation between two spectrally separate and spatially overlapping fluorescence detection channels can be used to detect the co-hybridization of two different hybridization probes labeled with spectrally-distinct fluorophores to target fragments. In contrast to single molecule fluorescence burst measurements, FCS can be used at higher concentrations of the fluorescent analyte (average probe volume occupancy>1) as long as the fluorescence fluctuations due to single fluorescent molecules are detectable.

Confocal microscopy in combination with laser epi-illumination may be used to attain femtoliter or smaller fluorescence/luminescence detection volumes. The small interrogation (probe) volume allows the detection of single molecule fluorescence excited with inexpensive, low-power, continuous-wave lasers. For 0.1 nM and higher sample concentrations, diffusion is sufficient for transport of analyte molecules into and out of the probe volume. For lower analyte concentrations, the sample throughput rate through the probe volume can be increased by scanning the probe volume through the stationary sample or by scanning or flowing the sample through a stationary probe volume.

Pulsed laser excitation and time-correlated single-photon counting methods may be used to discriminate against background photons due to solvent Raman and Rayleigh scattering of the excitation laser. This allows the use of roughly 1000× larger, ˜picoliter size, interrogation volumes for single molecule luminescence detection. Analyte molecules are carried through the probe volume in a flowing sample stream.

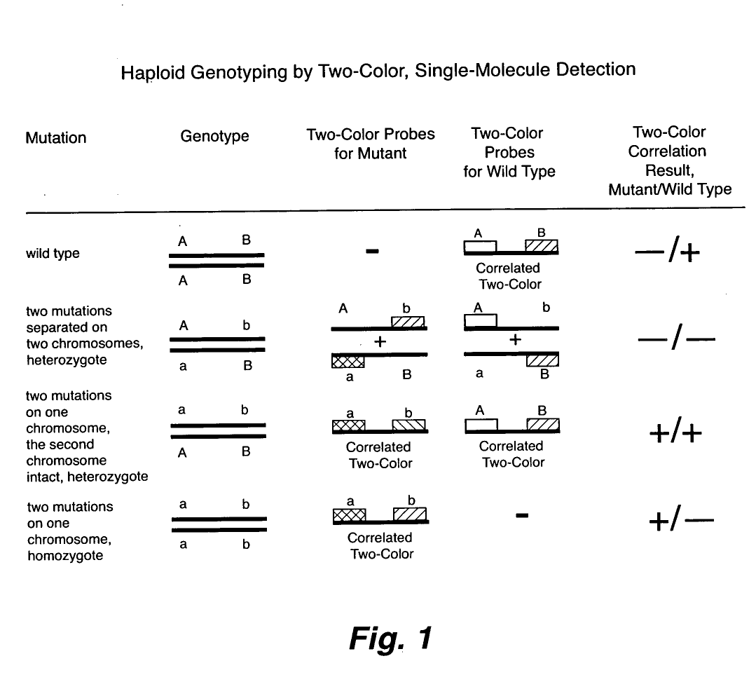

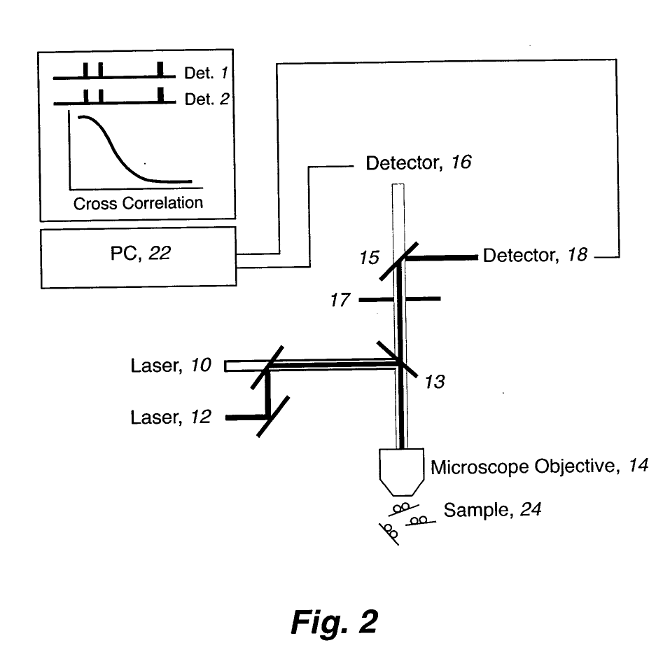

With reference to FIG. 1, the first step in this assay is to label the two genetic marker target sites, e.g., SNPs, with distinguishable, luminescent hybridization probes for the genetic targets. Suitable types of probes include single dye molecules, energy transfer dye pairs, nano-particles, luminescent nano-crystals, and intercalating dyes that fluorescence only after binding to a target. An ultra-sensitive, luminescence confocal microscope (exemplary detection apparatus), shown in FIG. 2, may be used to examine a dilute solution of the labeled DNA. Luminescence emission from two luminescent probes that are hybridized to single DNA fragments are recorded in two separate detection channels. The co-localization of the two hybridization probes on the same DNA haploid is signaled by the simultaneous detection of luminescence in both channels. By performing a cross-correlation between the detectors, one determines whether the DNA illuminated by the light beams contains one or both hybridization probes.

While FIG. 2 illustrates only the use of two luminescent probes with two corresponding detectors, it will be appreciated that more distinct probes might be used to simultaneously detect more genetic marker sites by inserting additional spectral filters and concomitant detectors for the wavelengths of interest. It is not intended to limit the present invention to only two probe types.

Haplotyping according to the present invention is applicable to a whole chromosome by sequentially and repeatedly typing pairs of neighboring genetic markers. For example, typing ten SNPs on a chromosome can be accomplished by typing two (or more) nearby SNPs (say SNP1 and SNP2) in the first experiment, SNP2 and SNP3 in the second experiment, SNP3 and SNP4 in the third experiment, and so forth.

In the example shown in FIG. 1 to determine the four haplotype combinations that characterize a genetic profile, two sets of polymorphism-specific probes that specifically bind to either the wild-type (A or B) or mutant (a or b) forms of two polymorphic sites of a gene are constructed. For example, one color probe may bind to (A and a) and a second color probe may bind to (B and b). As shown in FIG. 1, all four of the possible haplotypes can be identified by using both sets of probes in a pair of two-color, single molecule assays.

A typical protocol using well-known procedures would form four probes: two “red” probes to specifically hybridize with (A and a) sequences and two “green” probes to specifically hybridize with (B and b) sequences. The terms “red” and “green” as used herein are not limited to colors, but conveniently distinguish two distinct probe characteristics that are being detected. A two stranded DNA segment is denatured to provide two single stranded segments and a set of probes is then hybridized to the single stranded segments. For example, (A and B) probes are first specifically hybridized to the segments and the results analyzed. In a parallel reaction, the (a and b) probes are specifically hybridized to the segments and the results analyzed. As used herein, the term “specifically hybridize” means that the probe will hybridize only to the exact complementary nucleotide sequence on the fragment being tested.

If only wild type sequences are on a single stranded segment, then only the first set of probes will produce an output to illustrate that A and B sequences are on all of the chromosomes. If a wild type and a mutant sequence are present on each chromosome, then there will be no two-color output since each segment will hybridize with only one probe for each probe set. If one chromosome contains only wild type sequences and another chromosome contains only a mutant sequence, then both sets of probes will produce two-color outputs. Finally, if both chromosomes contain only mutant type sequences, then only the (a and b) probe set will provide a two-color output. Thus, the four probes generate a complete haplotype of the chromosome.

It should be noted that a single haplotype requires only the identification of two polymorphisms in a region of a chromosome. In the above example, only two probes might be used if a specific pair of polymorphisms, e.g., A and b, a and B, A and B, or a and b, is being screened.

The proposed single molecule haplotyping approach has a number of advantages over current haplotyping methods. These are:

(1) Easy interpretation. The single molecule approach is a direct haplotyping method. No inferred analysis is needed.

(2) Ability to type large regions (˜200 kb or more) in a single assay. Since the typing is performed directly on the chromosome, the length of the chromosome fragment that be can be typed is limited only by mechanical shearing of the genomic DNA during handling. In principle, the whole chromosome can be haplotyped by sequentially typing every two neighboring SNP markers with one common overlapping SNP. This will be significantly simpler than PCR-based typing, which is limited to a few kilobases between targets.

(3) High sensitivity. The assay will require only 20 ng or less of genomic DNA, about the same amount needed for a typical PCR reaction. No amplification of sample DNA is required prior to analysis.

(4) High throughput. The confocal microscope arrangement used for fluorescence detection can easily be adapted to a sample droplet array format for high throughput analysis.

(5) Less likely to have a false positive as a result of contaminant amplification. In a typical haplotyping, target amplification is required, such as PCR, which is very sensitive to sample contamination. For instance, a sample contaminated with a few copies of human DNA can be amplified and typed. As a result, a false positive or wrong haplotype may be obtained. Direct typing on human genomic DNA avoid the amplification of contamination, thus the typing result is less likely to be affected by a small amount of contamination.

Detection Schemes:

A schematic of exemplary apparatus capable of two-color single molecule lumisence detection is shown in FIG. 2. Laser epi-illumination is used in combination with confocal fluorescence detection to probe an extremely small volume of the solvent. Two excitation lasers 10, 12 are focused through microscope objective 14 to simultaneously excite DNA sample 24 that has been labeled with one or two distinguishable fluorophores. For a very dilute DNA solution, no DNA fragments will be inside the focused laser beam most of the time. When an individual DNA strand diffuses into the excitation region, the fluorophore labels on the DNA will fluoresce. The fluorescence is collected with microscope objective 14, passes through polychroic beam splitter 13, and spectrally split with dichroic beam splitter 15 between two sensitive photon counting detectors 16, 18. DNA strands that contain both fluorophores will be registered in both detectors. DNA with only one label will be seen by a single detector 16 or 18. The intensity recorded by each detector 16, 18 is cross-correlated to detect the presence of DNA fragments containing both labels.

An exemplary apparatus is based on a known laser epi-illuminated and confocal fluorescence emission collection design. The linear dimensions of the probe volume for the sample 24 are on the order of a micron or less resulting in a probe volume on the order of 1 femtoliter (fL) (Rigler, et al. 1993). Laser 10 is an Ar⁺ laser operating at 496 nm to excite a fluorescein fluorophore. Laser 12 is a helium neon laser operating at 633 nm to excite the fluorophore N,N′-biscarboxypentyl-5,5′-disulfonatoindodicarbocyanine (Cy5). Detectors 16 and 18 are single photon counting avalanche photodiodes. The detection channel from detector 16 is band pass filtered (filter not shown) to detect, e.g., green fluorescein emission. The detection channel from detector 18 is band pass filtered (filters not shown) to detect, e.g., red Cy5 emission. A pinhole 17 in the image plane of microscope objective 14 limits the field of view of two detectors 16, 18 to the immediate vicinity of the overlapping, focused laser beams. Sample 24, a microliter drop (e.g., 5 microliters) of a dilute solution of fluorescently labeled DNA in this exemplary apparatus, is suspended on the underside of a microscope coverslip. The coverslip is mounted on a scanning stage to allow the fluorescence detection probe volume to be raster scanned through the volume of the sample droplet. A personal computer (PC) 22 houses a commerically available digital correlator card (ALV 5000/E) that computes the cross-correlation between the two detection channels in real-time.

When an individual DNA fragment in sample 24 diffuses into the excitation region defined by microscope objective 14, the fluorescently-labeled probes on the DNA fragment will fluoresce. The fluorescence is collected and spectrally split between two sensitive detectors 16, 18. Signals from DNA fragments that contain both probes will be registered in both detectors. A signal from a DNA fragment with only one hybridization probe will be registered by only a single detector.

The intensity recorded by each detector is cross-correlated by computer 22 to look for instances where both probes were present on the same DNA fragment. In cross-correlation analysis, the time history of the fluorescence intensity recorded by one of the detectors (e.g., a red detector), Fᵣ(t), is multiplied with that recorded by the second detector (e.g., a green detector), F_{g}(t), at some later time, τ. This multiplication is averaged over all of the measurement time and the result is normalized and displayed as a function of the delay time, τ, between the two detectors. (See Equation 1):
G_{rg}(τ)α<δFᵣ(t)δF_{g}(t+τ)> Eq (1)

In the above expression, the brackets denote a time average, δ the deviation from the average value (i.e. δF(t)=F(t)−<F>), and G_{rg}(τ) is the cross-correlation between, e.g., the green and red fluorescence signals.

When individual DNA fragments labeled with both “red” and “green” fluorophores cross the excitation laser beam, fluorescence is recorded by both detectors and a non-zero cross-correlation between the detectors results. In this situation, the cross-correlation function should peak at τ=0 (i.e., zero delay time) and decay with a time constant that is roughly equal to the diffusion time of the DNA through the laser beam. Fluorescence from DNA fragments labeled with only a single color are detected in only one of the two channels and do not contribute to the cross correlation.

In practice, the sensitivity of the above analysis is limited by cross-talk between the green and red detection channels due to fluorescence from the red tail of the green fluorophore that overlaps the detection bandpass of the red detector. With currently available fluorophore pairs the cross-talk of the green emission into the red detector is ˜1% of that detected in the green channel. This limits the detection sensitivity of the doubly-labeled adduct to ˜1% of the green labeled species (Schwille et al. 1997). To avoid this problem, the data from the two detectors can be searched for photon bursts and the burst detection times from the red and green detectors can be cross correlated (Castro et al. 1997). The threshold for burst detection in the red channel can be adjusted to eliminate cross-talk between the green and red detection channels. This analysis requires that the average occupancy of fluorescent species in the probe volume be <<1, that is, the sample must be extremely dilute.

Another issue is that of unbound fluorescent probe. In the two-color FCS analysis described above, the amplitude of the cross-correlation, G_{rg}(τ), is proportional to the concentration of the double-labeled target DNA and inversely proportional to the product of the concentrations of the ‘green’ and ‘red’ probes. Therefore, excessively high concentrations of unbound probes will give small cross-correlation amplitudes and will reduce the accuracy of the measurement. If necessary, excess unbound probe can be removed from the sample prior to analysis using standard separation techniques such as, column chromatography (e.g., size exclusion and ion exchange) and electrophoresis.

The technique, described above, illustrates a detection capability for two hybridization probes located on the same or different DNA strands. However, the method as outlined suffers from the drawback that it can take a few milliseconds for the DNA in solution to diffuse into and out of the laser beam. For dilute DNA solutions, the excitation laser beams will be probing empty solvent most of the time. Consequently, with extremely dilute (<1 pM) DNA solutions, excessively long interrogation times would be necessary to determine whether or not two hybridization probes were on the same or different DNA strands.

This problem has been circumvented (Castro et al. 1997) by flowing the sample through the excitation laser beam at rates approaching ˜100 microns/sec and by probing a relatively large volume of solvent. The combination of these techniques allowed a rapid (˜800 seconds) determination of whether or not two fluorescent hybridization probes were co-hybridized to single DNA fragments with excellent signal to noise at DNA fragment concentrations below 10 fM. The signal to noise demonstrated in this technique could be sacrificed to allow shorter analysis times.

Alternate solutions are to move the sample through a stationary probe volume or to move the prove volume through a stationary sample. By using scanning speeds of ˜5 mm/second and ˜50 femtomolar concentration of labeled DNA, analysis times on the order of ˜1000 seconds can determine whether or not two fluorecent hybridization probes are on the same or separate DNA strands.

This approach can be used for single molecule haplotyping. However, the apparatus is equipment intensive and is not suitable for clinical research. In order to detect single fluorescent molecules in relatively large (1 pL) probe volumes, a pulsed excitation laser and time gated single photon detection have been used (Ambrose et al. 1999). In time gated detection, photons that arrive coincident with the laser pulse are ignored, as these are mainly due to Raman and Rayleigh scatter of the solvent. Scattered light background can be suppressed by approximately two orders of magnitude using pulsed excitation and time-gated detection. Time-gating has been successfully employed for many single molecule studies.

### Proof of Principle Protocol

A two-color, single molecule fluorescence detection apparatus based on a previous design (Schwille 1997) was used to detect the simultaneous presence of two different, single fluorophore labels on DNA fragments. To demonstrate that two different fluorophore labels can be detected on a single DNA strand, M13mp18, a single-stranded, circular DNA 7249 bases in length was used as target DNA for the preliminary experiments described here. M13mp18 DNA contains single EcoR I and Hind III restriction sites at base positions 6230 and 6281, respectively. Two fluorescently labeled DNA oligonucleotide hybridization probes were purchased from a commercial oligonucleotide synthesis service. These were further purified by polyacrylamide gel electrophoresis.

The first probe was a 20-mer complementary to a 20 base region of the M13mp18 target containing the EcoR I restriction site. This oligonucleotide was labeled at its 5′ end with a single carboxyfluorescein (FAM) fluorophore. The sequence of this oligonucleotide is 5′FAM-gctcgaattcgtaatcatcg-3′ [SEQ ID NO: 1]. The base sequence comprising the EcoR I restriction site in the FAM-labeled probe is shown in bold type.

The second hybridization probe was an 18-mer complementary to an 18 base region of the M13mp18 target containing the Hind III restriction site and had the sequence 5′Cy5-cagtgccaagcttcgatg-3′ [SEQ ID NO: 2]. This oligonucleotide was labeled at its 5′ end with a single N,N′-biscarboxypentyl-5,5′-disulfonatoindodicarbocyanine (Cy5) fluorophore. The base sequence comprising the Hind III restriction site contained in the Cy5-labeled probe is shown in bold type.

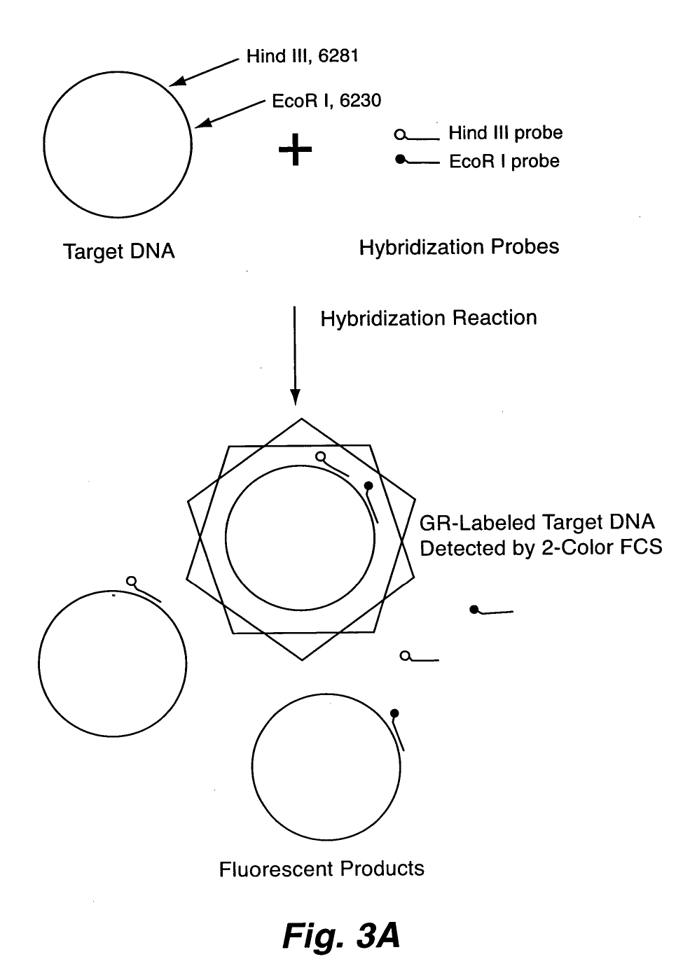

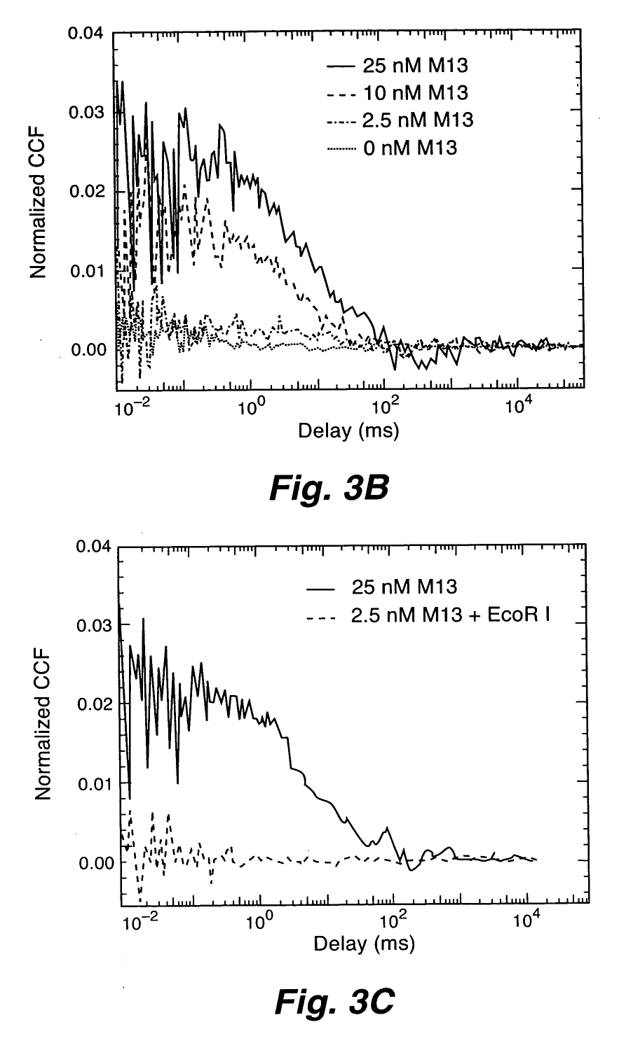

The hybridization reaction between the fluorescently labeled probes and the M13mp18 target DNA is shown schematically in FIG. 3A. The fluorescent products resulting from this reaction are also shown in FIG. 3A. The doubly-labeled probe/target adducts generate correlated fluorescence signals in the red and green detection channels and are detected by cross-correlation analysis. The singly-labeled fluorescent products do not produce correlated fluorescence signals in the red and green detection channels.

The samples consisted of 5 nM each of the fluorescently tagged oligonucleotide probes in a buffer containing 100 mM NaCl, 50 mM Tris pH 7.9, 10 mM MgCl2, 1 mM dithiothreitol and either 1 mg/ml or 0.1 mg/ml sheared salmon sperm DNA. Salmon sperm DNA was added to minimize non-specific binding of the target DNA and hybridization probes to surfaces during preparation and analysis of the samples. Target DNA concentrations were in the range of 2.5 nM to 25 nM. Negative controls included the probes without the M13mp18 template DNA as well as probes and template DNA digested with 10-20 units of EcoR I restriction enzyme to cut the hybridized FAM-probe/M13mp18 template adduct at the EcoR I restriction site in order to separate the FAM probe from the template. Hybridization reactions were carried out as follows. Samples were heated in a thermal cycler to 92° C. for a few minutes to denature double-stranded to single-stranded DNA and then cooled 50° C. and held at that temperature overnight to allow the complementary sequences (i.e., probes and template) to specifically hybridize to one another. When applicable, the samples were cooled to 37° C. at which point the EcoR I restriction enzyme was added. The restriction enzyme digestion was carried out for 2 hours at 37° C.

Samples were analyzed by two-color fluorescence cross-correlation using the apparatus shown in FIG. 2. A small amount (˜5 μL) of sample was pipetted onto a #1 borosilicate glass coverslip. The coverslip with the hanging sample droplet was placed under the microscope objective of the two-color fluorescence detection apparatus and the fluorescence excitation volume was positioned within the sample droplet ˜10 micrometers below the coverslip. Unless otherwise noted, cross-correlations were calculated from 600 s of fluorescence data collected from each sample. The samples were analysed at room temperature.

FIG. 3B shows cross-correlations obtained from samples containing different concentrations of the M13mp18 target DNA ranging from 0 nM to 25 nM. Each of these samples contained 1 mg/ml of sheared salmon sperm DNA. The cross-correlation from the sample containing no target DNA (dotted line) was calculated from 1800 s of fluorescence data. Under these experimental conditions, good cross-correlation was obtained from the sample containing 2 nM M13mp18 target and two hybridization probes.

FIG. 3C shows cross-correlations obtained from samples containing 25 nM M13mp18 target DNA (solid line), and 25 nM M13mp18 target DNA digested with EcoR I (dashed line). Each of these samples additionally contained 0.1 mg/ml of sheared salmon sperm DNA. The cross-correlation obtained from the sample that contained doubly labeled M13 shows substantial amplitude out to a delay of 100 ms. In contrast, the cross-correlation obtained from the EcoR I restricted sample (dashed line) shows very little amplitude. The EcoR I restriction enzyme specifically cuts at the six-base recognition site located within the duplex region formed by the hybridization of the FAM-labeled probe to its target sequence on the M13mp18 DNA. Once cut, the short FAM-labeled probe fragment dissociates from the M13mp18 DNA to effectively eliminate doubly-labeled target DNA adducts from the sample.

In the present invention, selected genetic marker sites would correspond to the Hind III and EcoR I restriction sites in the above experiment.

Target-Specific Probe Design.

A single probe approach refers to one probe that is targeted to one SNP. Compared with a multiple probe approach such as two-probe approach, where two probes are used to define one SNP, a single probe is economical and straightforward. Since most human SNPs are bi-allelic (only two alternative bases for each probe), SNP typing only needs to discriminate two bases at a given SNP site. DNA, PNA, molecular beacons, and LNA are possible probes for SNP analysis.

(1) Exemplary DNA and PNA Probes:

### EXAMPLE 1

### Leukemia Chimera Detection

In abnormal circumstances (such as some cancers), segments of genes may be recombined together, forming a so called chimera gene that is composed of incomplete parts of two genes. Traditionally, these hybrid genes are detected in a three step process using reverse transcriptase polymerase chain reaction (RT-PCR) amplification of mRNA; PCR amplification and DNA sequencing; and northern/southern blots or minisequencing. In accordance with the present invention, the amplification steps are eliminated and the two halves of the chimera gene messenger RNA are simultaneously detected using differently labeled fluorescent hybridization probes and fluorescence correlation spectroscopy (FCS). Since single molecule haplotyping can distinguish between a double and a single label, this method can distinguish between the chimera, which will be doubly labeled, and the singly labeled wild type transcripts present on separate messenger RNA molecules.

One example of a chimera, which is diagnostic of an acute lymphoblastic leukemia, is the MLL (HRX, Htrx) and AF4 (FEL) gene fusion. It is planned to use this system to develop methods to detect such chimera genes using FCS. Preliminary experiments will be conducted on a synthetic chimera template with sequence derived from both genes:

MLL-AF4/98(+)

[SEQ ID NO: 3]

Biotin-TEG 5′-gaagttcccaaaaccactcctagtgagcccaagaaa
aagcagcctccaccaccaaaacaatatgatacatcttcaaaaactcactc
aaattctcagc-3′.

Bold type indicates the sequence derived from the MLL gene; plain type indicates the sequence derived from the AF4 gene. A 5′ biotin is added to aid in troubleshooting, if necessary (see below). There is also a synthetic oligonucleotide complementary to the chimera synthetic template sequence above, designated MLL-AF4/98(−).

Fluorescently labeled DNA, peptide nucleic acid (PNA), and locked nucleic acid (LNA) oligonucleotides complementary to the sequences above are used as potential hybridization probes.

1. DNA probes sequences, including a linker of five extra dATPs follow:

MLL 3968L20

[SEQ ID NO: 4]

5′[Cy5]aaaaatttcttgggcttcactagggag-3′
AF4 4025L24

[SEQ ID NO: 5]

5′[FAM or Rhodamine Green X]aaaaaaatttgagtgagttttt
gaagatg-3′

2. PNA probe sequences. O stands for a linker sequence required between label and base.

MLLCy5P

[SEQ ID NO: 6]

(N)5′-[Cy5]-OOtttcttgggctcO-3′(C)
AF4FAMP

[SEQ ID NO: 7]

(N)5′-[FLU]-OOtttgagtgagttOlys-3′(C)

3. LNA probe sequences, locked nucleic acids bolded.

MLLCy5L

[SEQ ID NO: 8]

5′[Cy5]-tttcttgggctc-3′
AF4RGXL

[SEQ ID NO: 9]

5′[Rhodamine Green X]-tttgagtgagtt-3′

(Look 1997; VanDongen (1999))

The probes are labeled with biotin to allow binding to microspheres for fluorescence detection by a commercial flow cytometer (FACS Calibur, BD). Since the exemplary flow cytometer (FACS Calibur, BD) is optimized for detection of fluorescence in the mid-visible, fluorescein labels are used for all these evaluation studies. As shown in FIG. 1, two sets of the probes are needed, one that is wild type specific and another one that is mutant specific.

The probes (including DNA, PNA and LNA forms) will be tested for specificity to chimeric templates that are immobilized on the microspheres (Cai, Kommander et al. 1998; Nolan, Cai et al. 1998; Cai et al. 2000, Genomics.)

### EXAMPLE 2

### HLA Haplotying

One example of a region where haplotyping is critical for successful organ transplants is the human leukocyte antigen (HLA) gene. The method of the present invention is used pairwise on a set of variant alleles in this case. Preliminary work will be conducted on a set of synthetic oligonucleotide templates (haplotypes) containing a subset of the relevant sequence variants, where the variant alleles are underlined and the space represents a sequence of bases, e.g., a kb in length, separating variant alleles in the templates, where the missing sequence is not needed to construct a probe:

A*02011/A/TT/GT

[SEQ ID NO: 10]

5′tggcagctcagaccaccaagcacaagtgggag

[SEQ ID NO: 11]

. . . gcggcccatgtggcggagcagttgagagcctacctggagggcac
gtgcgtggagtggctccgcagatacctggaga-3′
A*0212/A/CA/GT

[SEQ ID NO: 12]

5′tggcagctcagaccaccaagcacaagtgggag

[SEQ ID NO: 13]

. . . gcggcccatgtggcggagcagcagagagcctacctggagggcac
gtgcgtggagtggctccgcagatacctggaga-3′
A*0236/A/TT/CG

[SEQ ID NO: 14]

5′tggcagctcagaccacccaagacaagtgggag

[SEQ ID NO: 15]

. . . gcggcccatgtggcggagcagttgagagcctacctggagggcac
gtgcgtggacgggctccgcagatacctggaga-3′
A*2402101/G/CA/CG

[SEQ ID NO: 16]

5′tggcagctcagaccacccaaggcaagtgggag

[SEQ ID NO: 17]

. . . gcggcccatgtggcggagcagcagagagcctacctggagggcac
gtgcgtggacgggctccgcagatacctggaga-3′
A*24031/G/CA/GT

[SEQ ID NO: 18]

5′tggcagctcagaccacccaaggcaagtgggag

[SEQ ID NO: 19]

. . . gcggcccatgtggcggagcagcagagagcctacctggagggcac
gtgcgtggagtggctccgcagatacctggaga-3′
A*2413/G/TT/GT

[SEQ ID NO: 20]

5′tggcagctcagaccacccaaggcaagtgggag

[SEQ ID NO: 21]

. . . gcggcccatgtggcggagcagttgagagcctacctggagggcac
gtgcgtggacgggctccgcagatacctggaga-3′

Target-specific fluorescent probes of DNA, PNA and LNA are designed to interrogate the sites of interest underlined above. The specificity of fluorescent probes will be tested on the above synthetic haplotype templates using flow methods described above.

(2) Molecular Beacon: Molecular beacons are oligonucleotide probes that fluoresce when they hybridize to their target. The hairpin shape of the molecular beacon causes mismatched probe/target hybrids to easily dissociate at significantly lower temperature than exactly complementary hybrids. This thermal instability of mismatched hybrids increases the specificity of molecular beacons. The presence or absence of a particular SNP sequence in DNA can be determined using a molecular beacon with a loop sequence complementary to a SNP target (Kostrikis et al., 1998; Tyagi et al., 1998). Exploiting the option to employ different dyes, molecular beacon assays can be multiplexed and have been used for real-time fluorescent genotyping (Kostrikis et al., 1998; Tyagi et al., 1998) and in the simultaneous detection of four different pathogenic retroviruses in clinical samples (Vet et al., 1999).

In single molecule haplotyping, two SNP specific molecular beacon probes are designed and labeled with two distinguishable fluorescent labels such as FAM and Cy5 using a standard protocol (Kostrikis et al., 1998; Tyagi et al., 1998). Upon simultaneous binding of the two beacon probes to a specific haplotype, the hairpins of two beacons will open up to release quenching of the fluorescence and a positive cross correlation of FAM and Cy5 will be detected and measured indicating the presence of that particular haplotype.

The main advantage of molecular beacons is that they remain nonfluorescent unless bound to a target. The fluorescence signal of a bound beacon compared to a free beacon can be as high as 500 fold (Tyagi 1998). The use of a beacon may enable single molecule haplotyping on unamplified genomic DNA without the separation of free probes. Furthermore, it was also reported that a beacon discriminates better than a DNA probe due to its rigid stem structure (Kostrikis et al. 1998).

(2) One Probe Labeling Protocol Using Peptide Nucleic Acid (PNA)

PNA is a unique class of informational molecule containing nucleobases attached to a neutral ‘peptide-like’ backbone (Egholm, M., 1993). PNA hybridizes to complementary RNA or DNA with higher affinity and specificity than conventional oligonucleotides and oligonucleotide analogues (Egholm, M., 1993, Wittung, P., 1994). The special properties of PNA allow novel molecular biology and biochemistry applications unachievable with traditional oligonucleotides and peptides (Buchardt, O.,1993). The backbone of the PNA molecule consists of repeating N-(2-aminoethyl) glycine units linked by amide bonds. The bases are attached to the backbone by methylene carbonyl linkages. Unlike DNA or other DNA analogs, PNA does not contain any pentose sugar moieties or phosphate groups. By convention, PNA is depicted like peptides, with the N-terminus at the first (left) position and the C-terminus at the right.

In single molecule haplotyping, two PNA probes labeled with two distingushable fluorophores (e.g., fluorophores available from Applied Biosystems) are designed to be specific for two SNPs of a particular haplotype. The presence of a specific haplotype is identified by the positive cross correlation of two fluorophores on the same chormosome. The protocol of PNA design, synthesis and the hybridization is known (Orum, et al. 1993; Castro, A. 1997).

It is expected that all the single probe biochemistry will work for this two SNP model system. However, PNA and molecular beacons are expected to be better choices, compared to a DNA probe, because of their greater discrimination capability between matched and mismatched bases. The replacement of a phosphodiester bond with a peptide bond makes PNA binding when paired with a matched DNA, but more unstable when bound to a mismatch. These properties of PNA have enabled PNA to be used in single point mutation detection through PCR clamping, a PCR amplification method of PNA-based single base discrimination assay (see, e.g., Orum et al. 1993). In addition, the single molecule detection of specific nucleic acid sequences in unamplified genomic DNA has been demonstrated using PNA probes(Castro 1997). All these results indicate that PNA-based sequence discrimination is highly specific. Therefore, the PNA probes are expected to be a good choice for single molecule haplotyping on genomic DNA.

(3). One Probe Approach-LNA

LNA (Locked Nucleic Acid) is a novel class of nucleic acid analogues. LNA monomers are bicyclic compounds structurally similar to RNA nucleosides (Koshkin 1998). The term “Locked Nucleic Acid” has been coined to emphasize that the furanose ring conformation is restricted in LNA by a methylene linker that connects the 2′-O position to the 4′-C position. For convenience, all nucleic acids containing one or more LNA modifications are called LNA. LNA oligomers obey Watson-Crick base pairing rules and hybridize to complementary oligonucleotides. LNA provides vastly improved hybridization performance when compared to DNA and other nucleic acid derivatives in a number of situations. LNA/DNA or LNA/RNA duplexes have increased thermal stability compared with similar duplexes formed by DNA or RNA. LNA has the highest affinity towards complementary DNA and RNA ever reported. In general, the thermal stability of a LNA/DNA duplex is increased 3° C. to 8° C. per modified base in the oligonucleotide. The design, synthesis and hybridization of LNA probes are known (Koshkin, 1998, Wahlestedt, 2000).

For single molecule haplotyping, LNA probes are designed, synthesized, and hybridized to SNPs according to the standard protocol in the references (Orum 1993; Orum 1999).

(4) FRET-Two Probe Approaches

Two hybridization probes, a probe bearing the fluorescence energy donor and an immediate adjacent probe bearing the energy transfer acceptor, i.e., energy transfer oligo pairs, are used for typing. The fluorophore of the donor may be the same for both SNPs, so a single excitation laser is used for both SNP targets. The acceptor fluorophores of two SNPs are distinguishable so that two color FCS analysis can be conducted. The presence of the particular haplotype is determined by a positive correlation of two color fluorescence as described above.

The synthesis and hybridization of fluorescent FRET probes is described in (Rasmussen et al. 1998). The fluorescence labels can be at 3′ or 5′ of an oligo depending on the numbers of nucleotides for the hybridization primers.

(5). Enzymatic Approach of Specific Labeling of SNPs.

Besides standard hybridization approaches (Landgren 1998), there have been numerous enzymatic labeling approaches that can be generally divided into polymerase-based (1-2), 3′-exonuclease-based (3), 5′-exonuclease-based (4), ligase-based (5) and endonuclease-based approaches (6-7). They can be a one primer approach or a two primer approache, or may be even combined. The respective protocols are known. (Syvanen 1990; Syvanen 1997; Davis et al. 1995; Lee. et al. 1993; Landegren et al. 1998; Lyamichev et al. 1999).

Other modified forms of enzyme-mediated approaches such as polymerase-mediated (Chen et al. 1999), ligase-mediated genotyping (Landegren et al. 1988; Landegren 1998), 5′-exonuclease (e.g. Taqman approach, Lee 1993) and Invader assay (Lyamichevet al. 1999) can also be labeled with FRET fluorophores and adapted for single molecule haplotyping purpose.

The potential limitation of a single probe can be one of the following:

(a) Optimization needed for each SNP. Since all above approaches are based on hybridization differences between a mismatched and matched base pairs, the thermal dynamic difference is small, especially for a T:G mismatch versus T:A match. To design a probe that is highly specific, experimental optimization may be needed even with the help of design guidelines and oligo analysis software.

(b) Routine experimentation may be needed to find a hybridization condition that would work for all probes in a large scale SNP analysis. There is a recent report showing multiplexed analysis of four targets in one reaction using four different molecular beacons (Vet et al. 1999). Adjusting the sequences of stems to make the beacon work at the desired temperature range is possible, but there may be some cases where the base discrimination only happens at a narrow range of temperature, and the hybridization condition for one SNP may not be the best for the second SNP. If this problem occurs, then the two probe designs such as oligo ligation and 5′ exonuclease may be used. Because multiple probes are required to define one SNP target, it generally has greater discrimination of the target over other genomic DNA than the one probe approach where only a single probe is required for each target as described in the two probe section.

The two-color, single molecule fluorescence detection laboratory apparatus can be optimized using the synthetic fluorescently-labeled oligo system and model hybridization probe systems derived from biochemistry research discussed above. Specific optimization parameters include the size of the excitation/detection volume, the excitation laser powers, and sample stage scanning rate for single molecule haplotyping. A synthetic fluorescently labeled oligo system may be used to explore the sensitivity of the apparatus to two-color labeled DNA fragments in the presence of a background of one-color labeled DNA. Data acquisition times and cross-correlation analysis routines can be optimized to give acceptably low assay error rates.

A number of hybridization probe schemes for haplotyping can be evaluated using conventional flow cytometry methods (Cai 1998; Cai et al. 2000.). The most promising of these can be tested with the single molecule fluorescence detection apparatus. Given the sensitivity of this apparatus, fluorescent hybridization probe schemes will likely have to be modified to minimize the introduction or retainment of fluorescent impurities that will degrade the single molecule fluorescence assay. Moreover, problems associated with non-specific binding of hybridization probes to genomic DNA, which become apparent only at the single molecule level, may arise.

Compared with the single probe approach, one advantage of multiple probes, such as a two-probe method, is the higher specificity of SNP targeting, because it requires the simultaneous hybridization of two probes to one SNP site. Unlike the conventional genotyping, where target sequences are amplified before typing, the high specificity of targeting may be needed for typing a specific target on the unamplified genomic DNA. As a matter of fact, the detection of a specific sequence on unamplified maize genomic DNA by two color single molecule technique has been done by using two PNA probes (Castro et al.1997). Another advantage of a two-probe method is that very little optimization is needed for the probes because the discrimination of matched and mismatched base pairs is dependent upon the intrinsic discrimination properties of enzymes such as ligase or 5′ exonuclease, not the hybridization of probes (e.g., reviewed by Landegren et al. 1998), i.e., all the target probing procedures can be done under one universal enzyme reaction condition. Therefore, the optimization of probes may be minimized and a universal hybridization reaction condition may be used for multiple SNP typing. This universal typing reaction condition allows the usage of robots to simultaneously process many samples.

(1) Modified Oligo ligation assay: A conventional ligase-based oligo ligation assay by flow cytometry for bulk genotyping purpose has been demonstrated (Landgren et al. 1998). However, the assay can not be directly used for the single molecule approach. The conventional ligation assay has a denaturation step to separate ligation product from bound (but not ligated ) probes, and to capture the ligation product for fluorescence detection. The denaturation, however, is not compatible with two-color haplotyping because two signals from one chromosome are detected and the denaturation would cause the dissociation of the probe from the target.

To adapt the oligo ligation assay to single molecule haplotyping, a modified ligation assay may be performed for two-color haplotyping. Two types of probes may be generated: one is an universal fluorescent reporter probe that binds to the 5′ end of a target, the other one is a blocker probe that has a specific base at the 5′ end (opposite to the SNP site) and a modified 3′ end (such as a carbon spacer). Upon the hybridization of two probes to a target, a ligase will join the matched blocker probe to the fluorescent reporter. Since the ligated fragment has a modified 3′ end that is resistant to Exonuclease III digestion, the fluorescence reporter will remain bound on the target. In contrast, if a blocker has a wrong base opposite a SNP target, a mismatch will be formed. Consequently, the ligation of the reporter and the blocker is prohibited, and Exonuclease III will invade from the nick and digest the fluorescent reporter. Therefore no fluorescence signal will be preserved on the DNA for two-color detection.

(2) Modified 5′ exonuclease assay (Taqman Assay): This is also an example of a two-probe approach that takes advantage of the 5′ exonuclease activity of Taq DNA polymerase to digest a DNA probe annealed at a SNP site. Typically, one primer binds to the upstream of the SNP target, and the other primer binds to the target with its 5′-terminus opposite to the SNP site. If the 5′ base of downstream primer B is perfectly matched with the SNP target, the polymerization continues. If the downstream primer is mis-paired with the SNP, then the polymerization is aborted at the SNP site. The 5′ exonuclease-based Taqman assay has been very successful for SNP genotyping (Holland et al. 1991; Landegren et al. 1998; Luthra 1998) and is adaptable to the single molecule haplotyping.

Two probes are prepared for each SNP target: upstream primer and a 3′-fluorescent downstream primer. Taq polymerase starts the polymerization from the 3′ end of upstream primer. When it encounters the matched downstream primer, the 5′ exonuclease digests away the downstream primer and the polymerization continues. Consequently there are no labels attached to the target. Note the 3′-end of the downstream primers is blocked with a fluorescein modification, and there is no polymerization on the downstream primer. On the other hand, if polymerase encounters a mismatched downstream primer, the 5′ exonucelase reaction is highly inhibited. As a result, the downstream fluorescent primer remains bound to a target. The typing of a SNP is based on the intrinsic discrimination against mismatch of 5′ exonucelase of Taq polymerase.

(3) Polymerase-Associated Proofreading Exonuclease/Ligation Assay:

Two primers are used for each SNP target: a 5′-labeled fluorescent upstream primer that binds to a SNP target and a 3′-amino-labeled (as a blocker of the exonclease which degrades DNA from a nick site) downstream primer that binds adjacent to 3′ of the target. If the upstream primer bears a correct 3′ base opposite target SNP, two primers will be joint by DNA ligase in the presence of DNA polymerase-associated proofreading exonucelase (a 3′-5′ exonucelase associated with a polymerase) and a additional exonuclease that degrades DNA from a nick. On the other hand, if the upstream primer bears a wrong base at the target site, polymerase-associated exonucelase will remove the wrong base and create a nick that is subjected to exonucelase degradation. As a result, fluorescent primer will be digested, and no fluorescence signal is given at the target site.

A potential limitation of the two probe approach is that a low signal-to-noise ratio originates from inefficient discrimination against the wrong bases. This can be intrinsic to ligase polymerase or exonuclease. However, data from available reports indicated that it should not be a big problem for most sequences (Holland et al. 1991; Lee et al. 1993; Luthra et al. 1998). If the target of leukemia and HLA do not permit a significant discrimination by a typical T4 phage ligase or Vent polymerase-associated 5′-exonuclease or other 3′ exonucleases, the design of the SNP specific primer can be improved to make the hybridization more specific to the mismatch, so the mismached primer will not bind to the target. Another alternative is to use ligases and 5′-exonucelases with higher discrimination ability from other species. For example, instead of T4 phage ligase, a thermalphile ligase can be used at higher temperatures, or E. Coli ligase, instead of Taq polymerase/exonuclease, or other polyemrases can be used.

In a typical pairing practice, the following parameters are evaluated and optimized for the single molecule haplotyping approach. The signal-to-noise level is obtained by comparing the positive signal of two-color correlation with a control experiment, where a restriction enzyme is added for cleavage between the two SNPs before the hybridization. Because the two SNPs are no longer physically linked, there should be no correlation between two SNPs.

Second, the amount of starting material needed for each assay is evaluated. This is important for future large-scale disease association analysis, because the ability for typing thousands of markers on the genomic DNA from each individual is crucial to find the link between markers and disease. In principle, the amount of DNA needed for the detection is less than one microliter at <10 fM. However, there is a practical concern for the loss of the DNA at each step of the assay. To evaluate the actual amount of DNA needed for each typing experiment, different amounts of starting blood or tissue samples are tested for each step of the assay, such as genomic DNA extraction and purification, probe hybridization, SNP typing reaction, separation of unbound probes from the bound probes and detection.

Third, the time needed for efficient binding to a target is evaluated. Based on the two color, single molecule hybridization study results from Castro et al. 1997, 14 hours of hybridization are needed at 100 pM of PNA probe concentration. This indicates an inefficient hybridization between the probes and an unamplified target at low concentrations. A time course study for the probe hybridization to the unamplified genomic DNA at different probe concentrations will determine the minimum time for the hybridization step. The efficient hybridization between probes and the target is optimized by exploring parameters such as salt concentrations, pH, temperature and addition of molecular crowding reagents. The time needed for single molecule detection also is optimized to increase the throughput of an assay. The data collection time is varied to determine the shortest collection time needed for a accurate typing.

### REFERENCES INCORPORATED HEREIN BY REFERENCE

Ambrose, W. P. et al. (1999). “Single molecule fluorescence spectroscopy at ambient temperature,” Chemical Reviews 99(10): 2929-2956.

Brookes A. J. et al (1999), “The essence of SNPs,” Gene 234:177-186.

Buchardt, O. et al. (1993) “Peptide nucleic acids and their potential applications in biotechnology” TIBTECH 11: 384-386.

Cai, H. et al. (1998). “Flow cytometry-based hybridization and polymorphism analysis,” Proc. SPIE 3256: 171-177.

Cai, H., et al (2000), “Flow cytometry-based minisequencing: A new platform for high throughput single nucleotide polymorphism scoring,” Genomics 66:135-143.

Castro, A. et al. (1997). “Single-molecule detection of specific nucleic-acid sequences in unamplified genomic DNA,” Analytical Chemistry 69(19): 3915-3920.

Chen X. N. et al. (1999) “Homogeneous genotyping assays for single nucleotide polymorphisms with fluorescence resonance energy transfer detection,” Genetic Analysis Biomolecular Engineering 14 (5-6): 157-163.

Davidson S. (2000) “Research suggests importance of haplotypes over SNPs” Nat. Biotech. 18:1134-1135

Davis, R. et al. (1995) “Method for detecting a nucleotide at a specific location within a nucleic acid using exonuclease activity,”. U.S. Pat. No. 5,391,480.

Drydale C. M. et al. (2000) “Complex promoter and coding region beta(2)-adrenergic recepter haplotypes alter receptor expression and predict in vivo responsiveness” PNAS 97:10483-10488.

Egholm, M., et al. (1993) “PNA hybridizes to complementary oligonucleotides obeying the Watson-Crick hydrogen bonding rules” Nature 365: 566-568.

Goodwin, P. M. et al. (1996). “Single-molecule detection in liquids by laser-induced fluorescence,” Accounts of Chemical Research 29(12): 607-613.

Hodge S. E. et al (1999). “Loss of information due to ambiguous haplotyping of SNPs”. Nat. Genetics. 21:360-361.

Holland, P. M. et al. (1991) “Detection of specific polymerase chain-reaction product by utilizing the 5′-3′ exonuclease activity of Thermus-Aquaticus DNA-polymerase, ” PNAS 88(16): 7276-7280.

Keller, R. A. et al. (1996) “Single-molecule fluorescence analysis in solution.” Applied Spectroscopy 50(7): A12-A32.

Koshkin, A. A. et al. (1998) “LNA (Locked Nucleic Acids): synthesis of the adenine, cytosine, guanine, 5-methylcytosine, thymine and uracil bicyclonucleoside monomers, oligomerisation, and unprecedented nucleic acid recognition” Tetrahedron 54: 3607-3630.

Kostrikis, L. G. et al. (1998). “Molecular beacons: Spectral genotyping of human alleles,” Science 279(5354): 1228-1229.

Kwok P. (1999). “Single nucleotide polymorphism libraries: why and how are we building them?” Molecular Medicine Today 5:538-543.

Landegren, U. et al. (1988) “A Ligase-mediated gene detection technique,” Science 241(4869): 1077-1080.

Landegren, U. et al. (1998). “Reading bits of genetic information : methods for single-nucleotide polymorphism analysis,” Genome Research 8(8): 769-776.

Lee, L. G. et al. (1993) “Allelic discrimination by nick-translation PCR with fluorogenic probes,” Nucleic Acids Research 21(16): 3761-3766.

Look, A. T. (1997) “Oncogenic transcription factors in the human acute leukemias” Science 278:1059-64.

Luthra, R. et al. (1998) “Novel 5′-exonuclease-based real-time PCR assay for the detection of T(14-18)(Q32-Q21) in patients with follicular lymphoma,” American Journal of Pathology 153(1): 63-68.

Lyamichev, V. et al. (1999) “Polymorphism Identification and quantitative detection of genomic DNA by invasive cleavage of oligonucleotide probes,” Nat. Biotechnol. 17: 292-296.

Nielsen, P. E. et al. (1991) “Sequence-selective recognition of DNA by strand displacement with a thymine-substituted polyamide,” Science 254: 1497-1500.

Orum, H. et al. (1993) “Single-base pair mutation analysis by PNA directed CR Clamping,” Nucleic Acids Research 21(23): 5332-5336.

Orum, H. et al. (1999) “Detection of the Factor V Leiden mutation by direct allele specific hybridization of PCR amplicons to photoimmobilized locked nucelic acids,” Clinical Chemistry 45:1898-1905.

Rasmussen, R. et al. (1998) “Quantitative PCR by continuous fluorescence monitoring of a double strand DNA specific binding dye,” Biochemica 2: 8-11.

Rigler, R. et al. (1993). “Fluorescence correlation spectroscopy with high count rate and low-background: analysis of translational diffusion,” Eur. Biophys. J. 22(3):169-175.

Ruano, G. et al. (1990) “Haplotype of multiple polymorphisms resolved by enzymatic amplfication of single DNA molecule” PNAS 87:6296-6300.

Syvanen, A. C. et al. (1990) “A primer-guided nucleotide incorporation assay in the genotyping of apolipoprotein-E,” Genomics 8(4): 684-692.

Syvanen, A. C. et al. (1997) “Minisequencing: a specific tool for DNA analysis and diagnostics on oligonucleotide arrays” Genome Res. 7:606-614.

Taton, T. A. et al. (2000) “Haplotyping by force.” Nature Biotechnology 18 713-713.

Tyagi, S. et al. (1998) “Multicolor molecular beacons for allele discrimination,” Nature Biotechnology 16(1): 49-53.

VanDongen, J. M. et al. (1999), “Standardized RT-PCR analysis of fusion gene transcripts from chromosome aberrations in acute leukemia for detection of minimal residual disease: Report of the BIOMED-1 Concerted Action: Investigation of minimal residual disease in acute leukemia,”Leukemia 13: 1901-28.

Vet, J. A. M. et al. (1999) “Multiplex detection of four pathogenic retroviruses using molecular beacons,” Proceedings of the National Academy of Sciences of the United States of America 96(11): 6394-6399.

Vogelstein, B. (1999) “Digital PCR,” PNAS 96(16): 9236-9241

Wahlestedt C. et al. (2000) “Potent and nontoxic antisense oligonucleotides containing locked nucleic acids, ” PNAS 97, 5633-5638.

Wittung, P. et al. (1994) “DNA-like double helix formed by peptide nucleic acid,” Nature 368: 561-563.

Wooley A. T. et al. (2000) “Direct haplotyping of kilobase-size DNA using carbon nanotubes probes,” Nature Biotechnology 18: 760-763.

Zhang L. (1992) “Whole genome amplification from a single cell: Implications for genetic analysis,” PNAS 89:5847-5851.

The foregoing description of the invention has been presented for purposes of illustration and description and is not intended to be exhaustive or to limit the invention to the precise form disclosed, and obviously many modifications and variations are possible in light of the above teaching. The embodiments were chosen and described in order to best explain the principles of the invention and its practical application to thereby enable others skilled in the art to best utilize the invention in various embodiments and with various modifications as are suited to the particular use contemplated. It is intended that the scope of the invention be defined by the claims appended hereto.

What is claimed is:

1. A method for rapid haplotyping comprising the steps of:
labeling at least two target sites on a segment of DNA or RNA with separate distinguishable luminescent hybridization probes, where the targets are selected genetic markers; and
detecting the presence or absence of each luminescent hybridization probe on each DNA segment to determine the haplotype of each DNA or RNA segment.

2. A method for rapid haplotyping comprising the steps of:
labeling at least two target sites on a segment of DNA or RNA with separate distinguishable luminescent hybridization probes, where the targets are selected genetic markers;
forming a dilute solution containing the labeled DNA or RNA segments;
illuminating each labeled DNA or RNA segment with light beams; and
detecting the presence or absence of each luminescent hybridization probe on each DNA segment to determine the haplotype of each DNA or RNA segment.

3. The method of claim 1, further including the step of sequentially and repeatedly haplotyping pairs of neighboring genetic makers on DNA segments forming a chromosome.

4. The method of claim 1, wherein the step of detecting the presence or absence of each luminescent hybridization probe includes the step of detecting a luminescence characteristic emitted from each hybridization probe and cross-correlating the detected luminescence characteristic to indicate the presence or absence of both hybridization probes on a single DNA or RNA segment.

5. The method of claim 2, wherein the step of detecting the presence or absence of each luminescent hybridization probe includes flowing the dilute solution of the DNA or RNA through a flow cytometer.

6. The method of claim 2, wherein the step of detecting the presence or absence of each luminescent hybridization probe includes examining individual drops of the solution containing DNA or RNA with a confocal microscope.

7. The method of claim 6, wherein the step of examining individual drops of the solution with a confocal microscope includes scanning each drop through a stationary probe volume or scanning the probe volume through a stationary drop.

8. The method of claim 1, 2, 3, 4, 5, 6, or 7, wherein the targets are base sequence variations selected from the group consisting of single nucleotide polymorphism, multibase deletion, multibase insertion, microsatellite repeats, di-nucleotide repeats, tri-nucleotide repeats, sequence rearrangements, and chimeric sequence.

9. The method of claim 1, 2, 3, 4, 5, 6, or 7, wherein the luminescent hybridization probes are selected to have distinguishable characteristics selected from the group consisting of luminescence emission spectral distribution, lifetime, intensity, burst duration, and polarization anisotropy.

10. The method of claim 9, wherein the luminescent hybridization probes are formed from the group consisting of single dye molecules, energy transfer dye pairs, nano-particles, luminescent nano-crystals, intercalating dyes, and molecular beacons.

11. The method of claim 9, wherein the form of the hybridization probe is selected from the group consisting of DNA, RNA, PNA, and LNA.

12. The method of claim 8, wherein the luminescent hybridization probes are selected to have distinguishable characteristics selected from the group consisting of luminescence emission spectral distribution, lifetime, intensity, burst duration, and polarization anisotropy.

13. The method of claim 12, wherein the luminescent hybridization probes are formed from the group consisting of single dye molecules, energy transfer dye pairs, nano-particles, luminescent nano-crystals, intercalating dyes, and molecular beacons.

14. The method of claim 12, wherein the form of the hybridization probe is selected from the group consisting of DNA, RNA, PNA, and LNA.

15. The method of claim 1, 2, 3, 4, 5, 6, or 7, where the hybridization probes are selected from the group consisting of single probes that are specific for each target or multiple probes that act together to identify the target.

16. The method of claim 15, wherein the single probes are selected from the group consisting of oligo DNA, oligo RNA, oligo beacon, oligo PNA, oligo LNA, and chimeric oligos.

17. The method of claim 15, wherein the multiple probes are selected from the group consisting of hybridization pairs, invader oligo pairs, ligation oligo pairs, mismatch extension 5′-exonuclease oligo pairs, energy transfer oligo pairs, and 3′-exonuclease pairs.

18. The method of claim 12, where the hybridization probes are selected from the group consisting of single probes that are specific for each target or two probes that act together to identify the target.

19. The method of claim 18, wherein the single probes are selected from the group consisting of oligo DNA, oligo RNA, oligo beacon, oligo PNA, oligo LNA, and chimeric oligos.

20. The method of claim 19, wherein the two probes are selected from the group consisting of hybridization pairs, invader oligo pairs, ligation oligo pairs, mismatch extension 5′-exonuclease oligo pairs, energy transfer oligo pairs, and 3′-exonuclease pairs.

21. The method of claim 1, 3, or 4 including forming a dilute solution to a concentration in the range of 100 nM to sub-fM of DNA or RNA fragments.


SEQUENCE LISTING (21 sequences)

SEQ ID NO: 1 - DNA, 20 bp, Unknown
  20-mer probe complementary to 20 base pair region of M13mp18 target containing EcoR I restriction site
gctcgaattcgtaatcatcg

SEQ ID NO: 2 - DNA, 18 bp, Unknown
  18-mer probe complementary to 18 base pair region of M13mp18 target containing Hind III restriction site
cagtgccaagcttcgatg

SEQ ID NO: 3 - DNA, 97 bp, Unknown
  Synthetic chimera template derived from genes MLL(HRX,Htrx) and AF4(FEL)
gaagttcccaaaaccactcctagtgagcccaagaaaaagcagcctccaccaccaaaacaatatgatacatcttcaaaaactcactcaaattctcagc

SEQ ID NO: 4 - DNA, 27 bp, Unknown
  DNA probe sequence complementary to MLL portion of SEQ ID NO3
aaaaatttcttgggcttcactagggag

SEQ ID NO: 5 - DNA, 29 bp, Unknown
  DNA probe sequence complementary to AF4 portion of SEQ ID NO3
aaaaaaatttgagtgagtttttgaagatg

SEQ ID NO: 6 - DNA, 12 bp, Unknown
  PNA probe sequence for MLL portion of SEQ ID NO3
tttcttgggctc

SEQ ID NO: 7 - DNA, 12 bp, Unknown
  PNA probe sequence for AF4 portion of SEQ ID NO3
tttgagtgagtt

SEQ ID NO: 8 - DNA, 12 bp, Unknown
  LNA probe sequence for MLL portion of SEQ ID NO3
tttcttgggctc

SEQ ID NO: 9 - DNA, 12 bp, Unknown
  LNA probe sequence for AF4 portion of SEQ ID NO3
tttgagtgagtt

SEQ ID NO: 10 - DNA, 32 bp, Unknown
  Synthetic oligonucleotide template containing a subset of a sequence variant for the HLA gene
tggcagctcagaccaccaagcacaagtgggag

SEQ ID NO: 11 - DNA, 76 bp, Unknown
  Synthetic oligonucleotide template containing a subset of a sequence variant for the HLA gene
gcggcccatgtggcggagcagttgagagcctacctggagggcacgtgcgtggagtggctccgcagatacctggaga

SEQ ID NO: 12 - DNA, 32 bp, Unknown; identical to SEQ ID NO: 10
  Synthetic oligonucleotide template containing a subset of a sequence variant for the HLA gene

SEQ ID NO: 13 - DNA, 76 bp, Unknown
  Synthetic oligonucleotide template containing a subset of a sequence variant for the HLA gene
gcggcccatgtggcggagcagcagagagcctacctggagggcacgtgcgtggagtggctccgcagatacctggaga

SEQ ID NO: 14 - DNA, 32 bp, Unknown
  Synthetic oligonucleotide template containing a subset of a sequence variant for the HLA gene
tggcagctcagaccacccaagacaagtgggag

SEQ ID NO: 15 - DNA, 76 bp, Unknown
  Synthetic oligonucleotide template containing a subset of a sequence variant for the HLA gene
gcggcccatgtggcggagcagttgagagcctacctggagggcacgtgcgtggacgggctccgcagatacctggaga

SEQ ID NO: 16 - DNA, 32 bp, Unknown
  Synthetic oligonucleotide template containing a subset of a sequence variant for the HLA gene
tggcagctcagaccacccaaggcaagtgggag

SEQ ID NO: 17 - DNA, 76 bp, Unknown
  Synthetic oligonucleotide template containing a subset of a sequence variant for the HLA gene
gcggcccatgtggcggagcagcagagagcctacctggagggcacgtgcgtggacgggctccgcagatacctggaga

SEQ ID NO: 18 - DNA, 32 bp, Unknown; identical to SEQ ID NO: 16
  Synthetic oligonucleotide template containing a subset of a sequence variant for the HLA gene

SEQ ID NO: 19 - DNA, 76 bp, Unknown; identical to SEQ ID NO: 13
  Synthetic oligonucleotide template containing a subset of a sequence variant for the HLA gene

SEQ ID NO: 20 - DNA, 32 bp, Unknown; identical to SEQ ID NO: 16
  Synthetic oligonucleotide template containing a subset of a sequence variant for the HLA gene

SEQ ID NO: 21 - DNA, 76 bp, Unknown; identical to SEQ ID NO: 15
  Synthetic oligonucleotide template containing a subset of a sequence variant for the HLA gene